(19) United States
(12) Patent Application Publication
(10) Pub. No.: US 2022/0193423 A1
(43) Pub. Date: Jun. 23, 2022

(54) CONNECTOR LOCKING ASSEMBLY FOR IMPLANTABLE PULSE GENERATOR

(71) Applicant: Advanced Neuromodulation Systems, Inc., Plano, TX (US)

(72) Inventors: Syed Askari-Raza Baqar, Frisco, TX (US); Robert Jones, McKinney, TX (US)

(21) Appl. No.: 17/214,513

(22) Filed: Mar. 26, 2021

Related U.S. Application Data
(60) Provisional application No. 63/128,448, filed on Dec. 21, 2020.

(51) Int. Cl. A61N 1/375; H01R 13/52; H01R 13/627

(52) U.S. Cl. CPC A61N 1/3752; A61N 1/3605; H01R 13/6278; H01R 13/5202

(57) ABSTRACT

An implantable pulse generator includes a housing component containing electrical circuitry for generating electrical pulses and a header component connected to the housing component. The header component is adapted to connect to one or more stimulation leads for applying the electrical pulses to the tissue of the patient. The header includes a locking member for an electrical terminal of the implantable pulse generator. The locking member includes a head, an elongate body portion adjacent the head and has a proximal section with a first diameter and a distal section having a second diameter. A transition section is between the proximal section and the distal section. The transition section smoothly transitions from the first diameter to the second diameter. The elongate body portion is configured to provide an interference fit to the electrical terminal in a locked position and hold the electrical terminal in place.

### PRIORITY

This application claims priority to U.S. Provisional Application No. 63/128,448, filed Dec. 21, 2020, the contents of which are hereby incorporated by reference in its entirety.

### FIELD

The present disclosure relates generally to a connector assembly for an implantable pulse generator for accepting one or more electrical connections for stimulation leads. More particularly, the present disclosure relates to locking members for the connector assembly.

### BACKGROUND OF THE INVENTION

Neurostimulation systems are devices that generate electrical pulses and deliver the pulses to neural tissue of a patient to treat a variety of disorders. One category of neurostimulation systems is deep brain stimulation (DBS). In DBS, pulses of electrical current are delivered to target regions of a subject's brain, for example, for the treatment of movement and effective disorders such as PD and essential tremor. Another category of neurostimulation systems is spinal cord stimulation (SCS) which is often used to treat chronic pain such as Failed Back Surgery Syndrome (FBSS) and Complex Regional Pain Syndrome (CRPS). Dorsal root ganglion (DRG) stimulation is another example of a neurostimulation therapy in which electrical stimulation is provided to the dorsal root ganglion structure that is just outside of the epidural space. DRG stimulation is generally used to treat chronic pain.

Neurostimulation systems generally include a pulse generator and one or more leads. A stimulation lead includes a lead body made of insulative material that encloses wire conductors. The distal end of the stimulation lead includes multiple electrodes, or contacts, that intimately impinge upon patient tissue and are electrically coupled to the wire conductors. The proximal end of the lead body includes multiple terminals (also electrically coupled to the wire conductors) that are adapted to receive electrical pulses. In DBS systems, the distal end of the stimulation lead is implanted within the brain tissue to deliver the electrical pulses. The stimulation leads are then tunneled to another location within the patient's body to be electrically connected with a pulse generator or, alternatively, to an “extension.” The pulse generator is typically implanted in the patient within a subcutaneous pocket created during the implantation procedure.

The pulse generator is typically implemented using a metallic housing (or can) that encloses circuitry for generating the electrical stimulation pulses, control circuitry, communication circuitry, a rechargeable or primary cell battery, etc. The pulse generating circuitry is coupled to one or more stimulation leads through electrical connections provided in a “header” of the pulse generator. Specifically, feedthrough wires typically exit the metallic housing and enter into a header structure of a moldable material. Within the header structure, the feedthrough wires are electrically coupled to annular electrical connectors. The header structure holds the annular connectors in a fixed arrangement that corresponds to the arrangement of terminals on the proximal end of a stimulation lead.

In known pulse generators, the header structure utilizes a small set screw that, when tightened, presses against a portion of the proximal end of the stimulation lead to hold the stimulation lead in the header. However, in some instances the set screws may become loose and the holding force against the stimulation lead is reduced, thus allowing for the stimulation lead to become inadvertently disconnected. In other instances, the set screws are very small and difficult for practitioners to properly position or tighten, which may in some circumstances lead to frustration or inadequate tightening of the set screw to the stimulation leads. In addition, the use of a set screw has required that the connection block be metal, which is undesirable during MRI conditions. In other instances, a user may forget to loosen the set screw before attempting to remove a stimulation lead, which may cause the damage to the IPG or the stimulation lead.

### BRIEF DESCRIPTION

In one embodiment an implantable pulse generator includes a housing component containing electrical circuitry for generating electrical pulses and a header component connected to the housing component. The header component is adapted to connect to one or more stimulation leads for applying the electrical pulses to the tissue of the patient. The header includes a locking member for an electrical terminal of the implantable pulse generator. The locking member includes a head, an elongate body portion adjacent the head and has a proximal section with a first diameter and a distal section having a second diameter. A transition section is between the proximal section and the distal section. The transition section smoothly transitions from the first diameter to the second diameter. The elongate body portion is configured to provide an interference fit to the electrical terminal in a locked position and hold the electrical terminal in place.

In another embodiment, a connection system for an implantable medical device (IMD) includes a connector block comprising a plurality of electrical contacts disposed sequentially with a through bore of the IMD, the through bore sized to accept a terminal of a stimulation lead, a septum, a first seal, and a locking pin. The locking pin comprises a head, an elongate body portion adjacent the head and having a proximal section with a first diameter and a distal section having a second diameter and a transition section between the proximal section and the distal section, the transition section smoothly transitioning from the first diameter to the second diameter. The elongate body portion is configured to provide an interference fit to the first seal when in a locked position and hold the terminal in place.

In yet another embodiment, a port plug for a connector block of an implantable medical device includes a plug head having a first diameter, an elongate body portion having a second diameter, and a shoulder disposed between the plug head and the elongate body portion, the shoulder having a third diameter larger than the second diameter.

### BRIEF DESCRIPTION OF THE DRAWINGS

FIG. 1 is a schematic view of a stimulation system according to an embodiment of the present disclosure.

FIG. 2 is a schematic view of an embodiment of a computing device of a stimulation device of the present disclosure.

FIG. 3 is a schematic view of an embodiment of a network environment for remote management of patient care according to the present disclosure.

FIG. 4 is a schematic view of an implantable pulse generator of an embodiment of the present disclosure.

FIG. 5 is a front view of an embodiment of a header of an implantable pulse generator of the present disclosure.

FIG. 6 is a partial isometric view taken from the front-left direction of the header of FIG. 5.

FIG. 7 is a partial isometric view taken from the front-right direction of the header of FIG. 5.

FIG. 8 is a cross sectional view of the header of FIG. 5

FIG. 9 is a front view of a pin according to an embodiment of the present disclosure.

FIG. 10 is a front view of a port plug within a header according to an embodiment of the present disclosure.

### DETAILED DESCRIPTION OF THE INVENTION

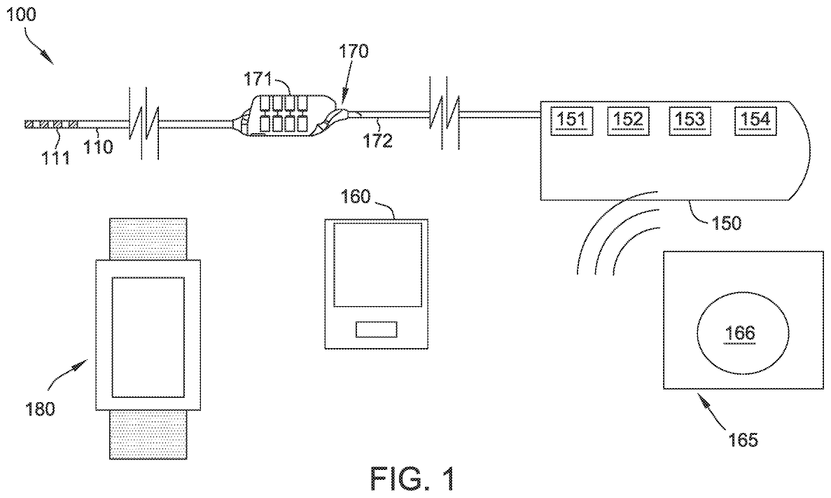

A stimulation system 100 is generally shown in FIG. 1 according to some embodiments. Stimulation system 100 generates electrical pulses for application to tissue of a patient to treat one or more disorders of the patient. System 100 includes an implantable pulse generator (IPG) 150 that is adapted to generate electrical pulses for application to tissue of a patient. Examples of commercially available implantable pulse generators include the PROCLAIM XR™ and INFINITY™ implantable pulse generators (available from ABBOTT, PLANO TX). Alternatively, in some embodiments, system 100 may include an external pulse generator (EPG) positioned outside the patient's body. IPG 150 typically includes a metallic housing (or “can”) that encloses a controller 151, pulse generating circuitry 152, a battery 153, far-field and/or near field communication circuitry 154 (e.g., BLUETOOTH communication circuitry), and other appropriate circuitry and components of the device. Controller 151 typically includes a microcontroller or other suitable processor for controlling the various other components of the device. Software code is typically stored in memory of IPG 150 for execution by the microcontroller or processor to control the various components of the device.

IPG 150 may comprise one or more attached extension components 170 or be connected to one or more separate extension components 170. Alternatively, one or more stimulation leads 110 may be connected directly to IPG 150. Within IPG 150, electrical pulses are generated by pulse generating circuitry 152 and are provided to switching circuitry. The switching circuit connects to output wires, metal ribbons, traces, lines, or the like (not shown) from the internal circuitry of pulse generator 150 to output connectors (not shown) of pulse generator 150 which are typically contained in the “header” structure of pulse generator 150. Commercially available ring/spring electrical connectors are frequently employed for output connectors of pulse generators (e.g., “Bal-Seal” brand connectors). The terminals of one or more stimulation leads 110 are inserted within connector portion 171 for electrical connection with respective connectors or directly within the header structure of pulse generator 150. Thereby, the pulses originating from IPG 150 are conducted to electrodes 111 through wires contained within the lead body of lead 110. The electrical pulses are applied to tissue of a patient via electrodes 111.

For implementation of the components within IPG 150, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference in its entirety. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference in its entirety.

An example and discussion of “constant current” pulse generating circuitry is provided in U.S. Patent Publication No. 2006/0170486 entitled “PULSE GENERATOR HAVING AN EFFICIENT FRACTIONAL VOLTAGE CONVERTER AND METHOD OF USE,” which is incorporated herein by reference in its entirety. One or multiple sets of such circuitry may be provided within IPG 150. Different pulses on different electrodes may be generated using a single set of pulse generating circuitry using consecutively generated pulses according to a “multi-stimset program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Stimulation lead(s) 110 may include a lead body of insulative material about a plurality of conductors within the material that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply stimulation pulses to tissue of the patient. Also, sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number and type of electrodes 111, terminals, and internal conductors.

External controller device 160 is a device that permits the operations of IPG 150 to be controlled by a user after IPG 150 is implanted within a patient. Also, multiple controller devices 160 may be provided for different types of users (e.g., the patient or a clinician). Controller device 160 can be implemented by utilizing a suitable handheld processor-based system that possesses wireless communication capabilities. In some embodiments, controller device 160 may be a smart phone or mobile electronic device configured to operate as controller device 160 described herein. Software is typically stored in a nontransitory memory of controller device 160 to control the various operations of controller device 160. The interface functionality of controller device 160 is implemented using suitable software code for interacting with the user and using the wireless communication capabilities to conduct communications with IPG 150. One or more user interface display screens may be provided in software to allow the patient and/or the patient's clinician to control operations of IPG 150 using controller device 160. In some embodiments, commercially available devices such as APPLE IOS devices are adapted for use as controller device 160 by include one or more “apps” that communicate with IPG 150 using, for example, BLUETOOTH® or other short range wireless communication systems.

Controller device 160 preferably provides one or more user interfaces to allow the user to operate IPG 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc.

Controller device 160 may permit programming of IPG 150 to provide a number of different stimulation patterns or therapies to the patient as appropriate for a given patient and/or disorder. Examples of different stimulation therapies include conventional tonic stimulation (continuous train of stimulation pulses at a fixed rate), BurstDR stimulation (burst of pulses repeated at a high rate interspersed with quiescent periods with or without duty cycling), “high frequency” stimulation (e.g., a continuous train of stimulation pulses at 10,000 Hz), noise stimulation (series of stimulation pulses with randomized pulse characteristics such as pulse amplitude to achieve a desired frequency domain profile). Any suitable stimulation pattern or combination thereof can be provided by IPG 150 according to some embodiments. Controller device 160 communicates the stimulation parameters and/or a series of pulse characteristics defining the pulse series to be applied to the patient to IPG 150 to generate the desired stimulation therapy.

Examples of suitable therapies include tonic stimulation (in which a fixed frequency pulse train) is generated, burst stimulation (in which bursts of multiple high frequency pulses) are generated which in turn are separated by quiescent periods, “high frequency” stimulation, multi-frequency stimulation, noise stimulation. Descriptions of respective neurostimulation therapies are provided in the following publications: (1) Schu S., Slotty P. J., Bara G., von Knop M., Edgar D., Vesper J. A Prospective, Randomised, Double-blind, Placebo-controlled Study to Examine the Effectiveness of Burst Spinal Cord Stimulation Patterns for the Treatment of Failed Back Surgery Syndrome. Neuromodulation 2014; 17: 443-450; (2) Al-Kaisy Al, Van Buyten JP, Smet I, Palmisani S, Pang D, Smith T. 2014. Sustained effectiveness of 10 kHz high-frequency spinal cord stimulation for patients with chronic, low back pain: 24-month results of a prospective multicenter study. Pain Med. 2014 March;15(3):347-54; and (3) Sweet, Badjatiya, Tan D1, Miller. Paresthesia-Free High-Density Spinal Cord Stimulation for Postlaminectomy Syndrome in a Prescreened Population: A Prospective Case Series. Neuromodulation. 2016 April; 19(3):260-7. Noise stimulation is described in U.S. Pat. No. 8,682,441B2. Burst stimulation is described in U.S. Pat. No. 8,224,453 and U.S. Published Application No. 20060095088. All of these references are incorporated herein by reference in their entireties.

In one embodiment, for implementation of the components within stimulation system 100, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference in its entirety. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION” which is incorporated herein by reference in its entirety.

In one embodiment, IPG 150 modifies its internal parameters in response to the control signals from controller device 160 to vary the stimulation characteristics of stimulation pulses transmitted through stimulation lead 110 to the tissue of the patient. Neurostimulation systems, stimsets, and multi-stimset programs are discussed in PCT Publication No. WO 2001/093953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference in their entireties.

External charger device 165 may be provided to recharge battery 153 of IPG 150 according to some embodiments when IPG 150 includes a rechargeable battery. External charger device 165 comprises a power source and electrical circuitry (not shown) to drive current through coil 166. The patient places the primary coil 166 against the patient's body immediately above the secondary coil (not shown), i.e., the coil of the implantable medical device. Preferably, the primary coil 166 and the secondary coil are aligned in a coaxial manner by the patient for efficiency of the coupling between the primary and secondary coils. In operation during a charging session, external charger device 165 generates an AC-signal to drive current through coil 166 at a suitable frequency. Assuming that primary coil 166 and secondary coil are suitably positioned relative to each other, the secondary coil is disposed within the magnetic field generated by the current driven through primary coil 166. Current is then induced by a magnetic field in the secondary coil. The current induced in the coil of the implantable pulse generator is rectified and regulated to recharge the battery of IPG 150. IPG 150 may also communicate status messages to external charging device 165 during charging operations to control charging operations. For example, IPG 150 may communicate the coupling status, charging status, charge completion status, etc.

System 100 may include external wearable device 180 such as a smartwatch or health monitor device. Wearable device may be implemented using commercially available devices such as FITBIT VERSA SMARTWATCH™, SAMSUNG GALAXY SMARTWATCH™, and APPLE WATCH™ devices with one or more apps or appropriate software to interact with IPG 150 and/or controller device 160. In some embodiments, wearable device 180, controller device 160, and IPG 150 conduct communications using BLUETOOTH® communications.

Wearable device 180 monitors activities of the patient and/or senses physiological signals. Wearable device 180 may track physical activity and/or patient movement through accelerometers. Wearable device 180 may monitory body temperature, heart rate, electrocardiogram activity, blood oxygen saturation, and/or the like. Wearable device 180 may monitor sleep quality or any other relevant health related activity.

Wearable device 180 may provide one or more user interface screens to permit the patient to control or otherwise interact with IPG 150. For example, the patient may increase or decrease stimulation amplitude, change stimulation programs, turn stimulation on or off, and/or the like using wearable device 180. Also, the patient may check the battery status of other implant status information using wearable device 180.

Wearable device 180 may include one or more interface screens to receive patient input. In some embodiments, wearable device 180 and/or controller device 160 are implemented (individually or in combination) to provide an electronic patient diary function. The patient diary function permits the patient to record on an ongoing basis the health status of the patient and the effectiveness of the therapy for the patient. In some embodiments as discussed herein, wearable device 180 and/or controller device 160 enable the user to indicate the current activity of the patient, the beginning of an activity, the completion of an activity, the ease or quality of patient's experience with a specific activity, the patient's experience of pain, the patient's experience of relief from pain by the stimulation, or any other relevant indication of patient health by the patient.

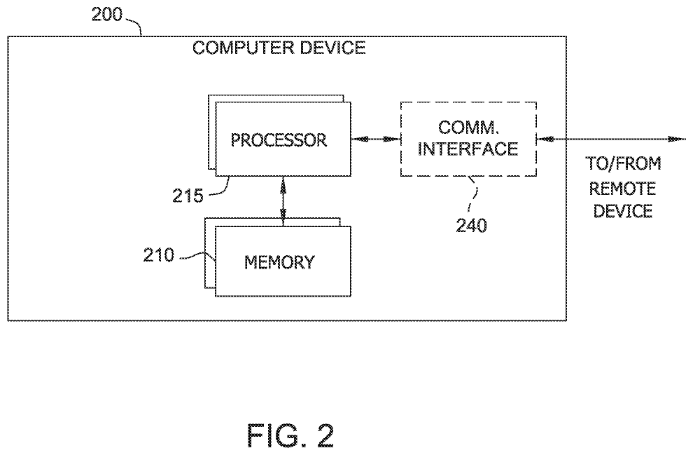

FIG. 2 is a block diagram of one embodiment of a computing device 200 that may be used to according to some embodiments. Computing device 200 may be used to implement external controller device 160, wearable device 180, remote care management servers, or other computing system according to some embodiments.

Computing device 200 includes at least one memory device 210 and a processor 215 that is coupled to memory device 210 for executing instructions. In some embodiments, executable instructions are stored in memory device 210, which may comprise a nontransitory memory. In some embodiments, computing device 200 performs one or more operations described herein by programming processor 215. For example, processor 215 may be programmed by encoding an operation as one or more executable instructions and by providing the executable instructions in memory device 210.

Processor 215 may include one or more processing units (e.g., in a multi-core configuration). Further, processor 215 may be implemented using one or more heterogeneous processor systems in which a main processor is present with secondary processors on a single chip. In another illustrative example, processor 215 may be a symmetric multi-processor system containing multiple processors of the same type. Further, processor 215 may be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein.

In the illustrated embodiment, memory device 210 is one or more devices that enable information such as executable instructions and/or other data to be stored and retrieved. Memory device 210 may include one or more computer readable media, such as, without limitation, dynamic random access memory (DRAM), read-only memory (ROM), electrically erasable programmable read-only memory (EEPROM), static random access memory (SRAM), a solid state disk, and/or a hard disk. Memory device 210 may be configured to store, without limitation, application source code, application object code, source code portions of interest, object code portions of interest, configuration data, execution events and/or any other type of data.

Computing device 200, in the illustrated embodiment, includes a communication interface 240 coupled to processor 215. Communication interface 240 communicates with one or more remote devices, such as a clinician or patient programmer. To communicate with remote devices, communication interface 240 may include, for example, a wired network adapter, a wireless network adapter, a radio-frequency (RF) adapter, and/or a mobile telecommunications adapter.

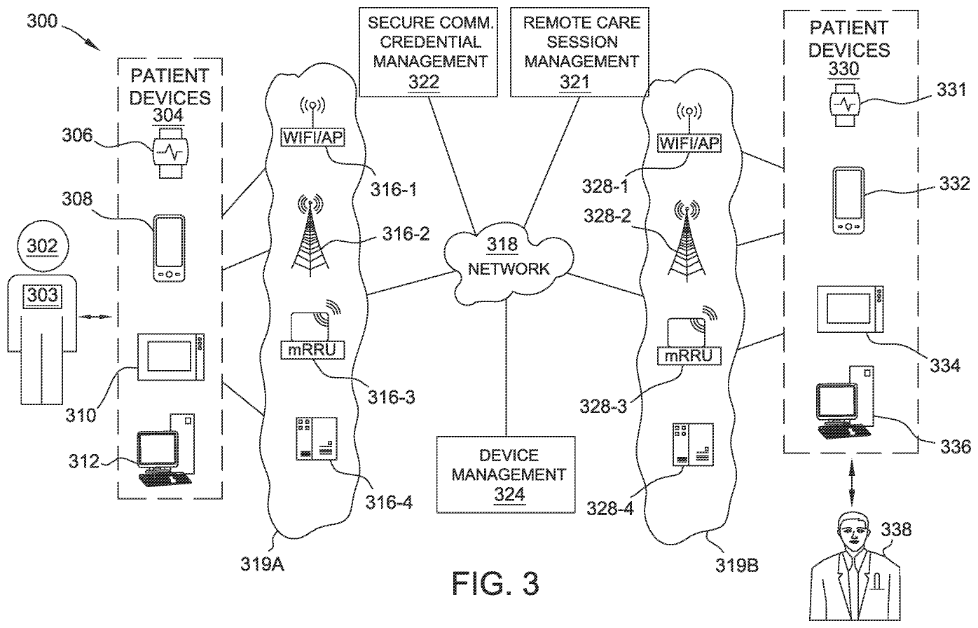

FIG. 3 depicts a network environment 300 for remote management of patient care. One or more embodiments of a remote care therapy application or service may be implemented in network environment 300, as described herein. In general, “remote care therapy” may involve any care, biomedical monitoring, or therapy that may be provided by a clinician, a medical professional or a healthcare provider, and/or their respective authorized agents (including digital/virtual assistants), with respect to a patient over a communications network while the patient and the clinician/provider are not in close proximity to each other (e.g., not engaged in an in-person office visit or consultation). Accordingly, in some embodiments, a remote care therapy application may form a telemedicine or a telehealth application or service that not only allows healthcare professionals to use electronic communications to evaluate, diagnose and treat patients remotely, thereby facilitating efficiency as well as scalability, but also provides patients with relatively quick and convenient access to diversified medical expertise that may be geographically distributed over large areas or regions, via secure communications channels as described herein.

Network environment 300 may include any combination or sub-combination of a public packet-switched network infrastructure (e.g., the Internet or worldwide web, also sometimes referred to as the “cloud”), private packet-switched network infrastructures such as Intranets and enterprise networks, health service provider network infrastructures, and the like, any of which may span or involve a variety of access networks, backhaul and core networks in an end-to-end network architecture arrangement between one or more patients, e.g., patient(s) 302, and one or more authorized clinicians, healthcare professionals, or agents thereof, e.g., generally represented as caregiver(s) or clinician(s) 338.

Example patient(s) 302, each having a suitable implantable device 303, may be provided with a variety of corresponding external devices for controlling, programming, otherwise (re)configuring the functionality of respective implantable medical device(s) 303, as is known in the art. Such external devices associated with patient(s) 302 are referred to herein as patient devices 304, and may include a variety of user equipment (UE) devices, tethered or untethered, that may be configured to engage in remote care therapy sessions. By way of example, patient devices 304 may include smartphones, tablets or phablets, laptops/desktops, handheld/palmtop computers, wearable devices such as smart glasses and smart watches, personal digital assistant (PDA) devices, smart digital assistant devices, etc., any of which may operate in association with one or more virtual assistants, smart home/office appliances, smart TVs, virtual reality (VR), mixed reality (MR) or augmented reality (AR) devices, and the like, which are generally exemplified by wearable device(s) 306, smartphone(s) 308, tablet(s)/phablet(s) 310 and computer(s) 312. As such, patient devices 304 may include various types of communications circuitry or interfaces to effectuate wired or wireless communications, short-range and long-range radio frequency (RF) communications, magnetic field communications, Bluetooth communications, etc., using any combination of technologies, protocols, and the like, with external networked elements and/or respective implantable medical devices 303 corresponding to patient(s) 302.

With respect to networked communications, patient devices 304 may be configured, independently or in association with one or more digital/virtual assistants, smart home/premises appliances and/or home networks, to effectuate mobile communications using technologies such as Global System for Mobile Communications (GSM) radio access network (GRAN) technology, Enhanced Data Rates for Global System for Mobile Communications (GSM) Evolution (EDGE) network (GERAN) technology, 4G Long Term Evolution (LTE) technology, Fixed Wireless technology, 5th Generation Partnership Project (5GPP or 5G) technology, Integrated Digital Enhanced Network (IDEN) technology, WiMAX technology, various flavors of Code Division Multiple Access (CDMA) technology, heterogeneous access network technology, Universal Mobile Telecommunications System (UMTS) technology, Universal Terrestrial Radio Access Network (UTRAN) technology, All-IP Next Generation Network (NGN) technology, as well as technologies based on various flavors of IEEE 802.11 protocols (e.g., WiFi), and other access point (AP)-based technologies and microcell-based technologies such as femtocells, picocells, etc. Further, some embodiments of patient devices 104 may also include interface circuitry for effectuating network connectivity via satellite communications. Where tethered UE devices are provided as patient devices 304, networked communications may also involve broadband edge network infrastructures based on various flavors of Digital Subscriber Line (DSL) architectures and/or Data Over Cable Service Interface Specification (DOCSIS)-compliant Cable Modem Termination System (CMTS) network architectures (e.g., involving hybrid fiber-coaxial (HFC) physical connectivity). Accordingly, by way of illustration, an edge/access network portion 119A is exemplified with elements such as WiFi/AP node(s) 316-1, macro/microcell node(s) 116-2 and 116-3 (e.g., including micro remote radio units or RRUs, base stations, eNB nodes, etc.) and DSL/CMTS node(s) 316-4.

Similarly, clinicians 338 may be provided with a variety of external devices for controlling, programming, otherwise (re)configuring or providing therapy operations with respect to one or more patients 302 mediated via respective implantable medical device(s) 303, in a local therapy session and/or remote therapy session, depending on implementation and use case scenarios. External devices associated with clinicians 338, referred to herein as clinician devices 330, may include a variety of UE devices, tethered or untethered, similar to patient devices 304, which may be configured to engage in remote care therapy sessions as will be set forth in detail further below. Clinician devices 330 may therefore also include devices (which may operate in association with one or more virtual assistants, smart home/office appliances, VRAR virtual reality (VR) or augmented reality (AR) devices, and the like), generally exemplified by wearable device(s) 331, smartphone(s) 332, tablet(s)/phablet(s) 334 and computer(s) 336. Further, example clinician devices 330 may also include various types of network communications circuitry or interfaces similar to that of patient device 304, which may be configured to operate with a broad range of technologies as set forth above. Accordingly, an edge/access network portion 319B is exemplified as having elements such as WiFi/AP node(s) 328-1, macro/microcell node(s) 328-2 and 328-3 (e.g., including micro remote radio units or RRUs, base stations, eNB nodes, etc.) and DSL/CMTS node(s) 328-4. It should therefore be appreciated that edge/access network portions 319A, 319B may include all or any subset of wireless communication means, technologies and protocols for effectuating data communications with respect to an example embodiment of the systems and methods described herein.

In one arrangement, a plurality of network elements or nodes may be provided for facilitating a remote care therapy service involving one or more clinicians 338 and one or more patients 302, wherein such elements are hosted or otherwise operated by various stakeholders in a service deployment scenario depending on implementation (e.g., including one or more public clouds, private clouds, or any combination thereof). In one embodiment, a remote care session management node 320 is provided, and may be disposed as a cloud-based element coupled to network 318, that is operative in association with a secure communications credentials management node 322 and a device management node 324, to effectuate a trust-based communications overlay/tunneled infrastructure in network environment 300 whereby a clinician may advantageously engage in a remote care therapy session with a patient.

In the embodiments described herein, implantable medical device 303 may be any suitable medical device. For example, implantable medical device may be a neurostimulation device that generates electrical pulses and delivers the pulses to nervous tissue of a patient to treat a variety of disorders.

Although implantable medical device 303 is described in the context of a neurostimulation device herein, those of skill in the art will appreciate that implantable medical device 303 may be any type of implantable medical device.

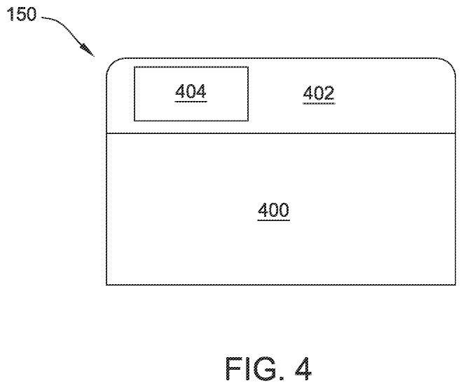

With reference to FIG. 4, in one embodiment, the implantable pulse generator 150 comprises a main body 400 and a header 402. In some embodiments, the main body 400 is metallic, but in other embodiments main body 400 may comprise other biocompatible materials such as plastic or the like. In one embodiment, the header 402 is removably securable to main body 400. The main body 400 is a housing that houses controller 151, pulse generating circuitry 152, a battery 153, far-field and/or near field communication circuitry 154 (e.g., BLUETOOTH communication circuitry), and other appropriate circuitry and components of the device connector assembly (FIG. 1). In one embodiment, the header 402 includes a connector block 404 therein.

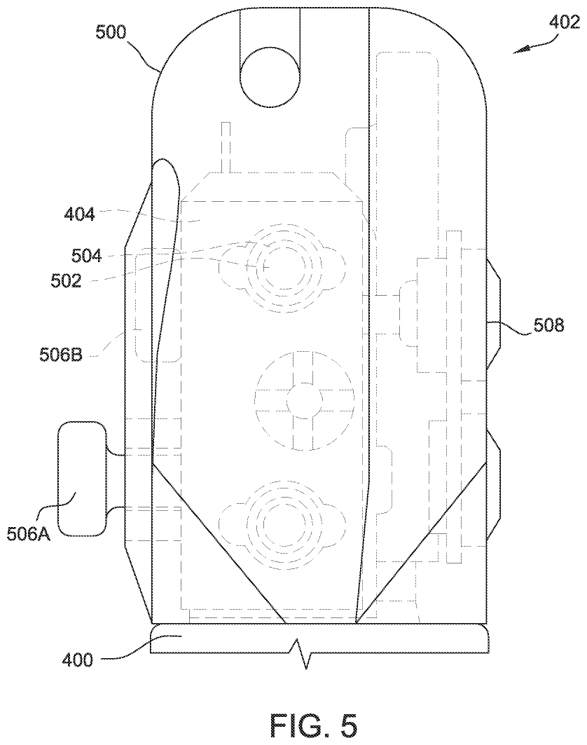

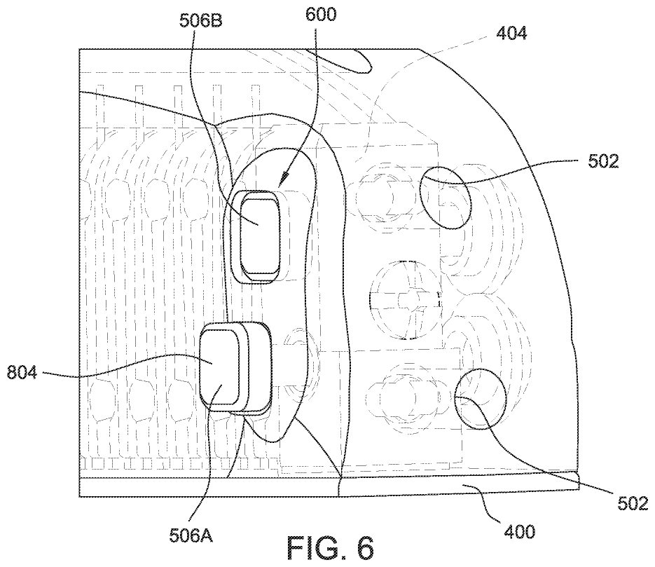

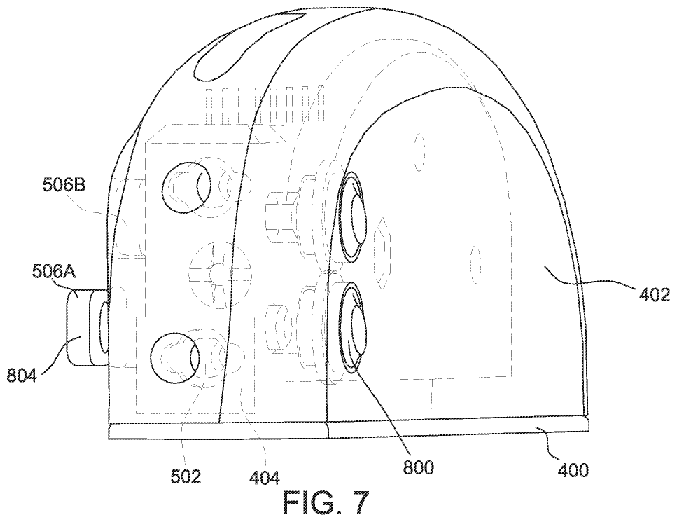

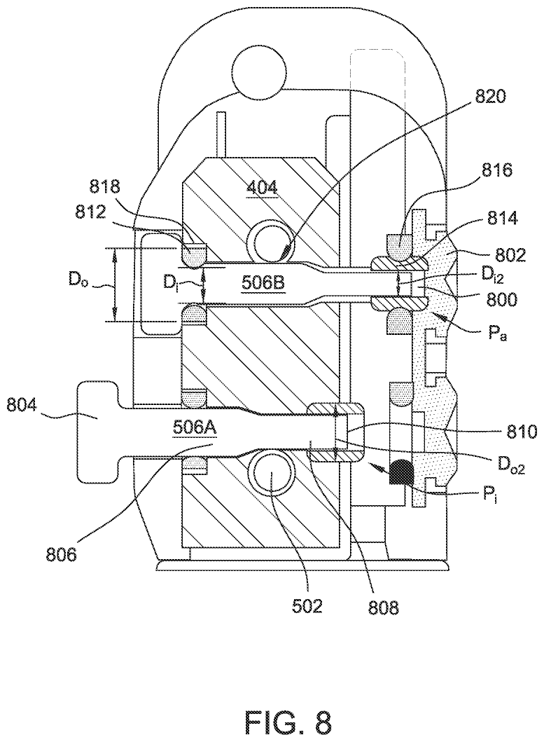

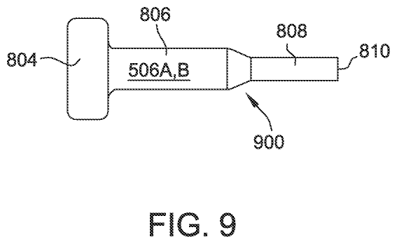

With reference to FIGS. 5-9, in one embodiment, header 402 includes an outer cover 500 that houses the connector block 404 therein. The connector block 404 includes one or more through bores 502 for accepting a corresponding terminal of a stimulation lead 110 (FIG. 1). The terminal at the proximal end of the stimulation lead comprises a conductive material for interfacing with electrical connectors 504 held within bores 502 the connector block 404. It is noted that in embodiments, the connector block 404 comprises a non-electrically conductive material. The electrical connectors 504 may be, for example ring shaped terminals such as Bal-Seal connectors, spring connectors or the like. In one embodiment, the bores 502 are configured to allow the terminals of the stimulation leads to be inserted therein. In one embodiment, the connector block 404 comprises at least two through-holes for accepting a terminal of a stimulation lead and at least two other through holes for engaging with the pins 506A,B. In embodiments, the electrical connectors 504 have a generally cylindrical shape with a plurality of inline contacts separated by insulating portions. Each terminal of the stimulation leads may include any number of inline contacts, such as ring-shaped contacts, separated by the insulated portions, such as six, eight, ten, twelve or any other number of inline contacts separated by insulating portions. Accordingly, a corresponding number of electrical connectors 504 may be present within the bores 502, separated by insulating material, to electrically connect to respective ones of the terminals of the stimulation leads. In embodiments, the header 402 includes one or more septums 508 that provide access to an interior of header 402. The septums 508 have a septum bore 800 (FIG. 8) sealed by a sealing member 802 which may comprise silicone or other biocompatible material capable of forming a liquid tight or hermetic seal.

Moreover, each terminal of the stimulation lead can have substantially any diameter. By way of example, the diameter of the terminal or the stimulation lead 110 can be from 0.025 to 0.1 inches, such as 0.050 inches, or any other diameter that allows the devices to operate as described herein. Correspondingly, the bores 502 of the terminal block are respectively sized to allow the terminal to fit therein.

In order to secure the terminals of the stimulation leads 110 within the connector block 404, one or more pins 506A,B are used to provide an interference fit to lock the terminals in place without the need for a set screw. Each of the pins 506A,B includes a head 804 and a pin body comprising a first body portion 806 and a second body portion 808. The first body portion 806 being adjacent to and proximal the head 804 and the second body portion 808 being adjacent to the first body portion 806 and distal to the head 804. The second body portion 808 terminates at end face 810. In one embodiment the first body portion 806 has a larger diameter than second body portion 808, and may include a tapered portion 900 between the first body portion 806 and second body portion 808. In some embodiments, the first body portion 806 and the second body portion are substantially cylindrical in shape. However in some embodiments, the first body portion 806 and the second body portion 808 may have a different shape, such as a square, oval, rectangular, polygonal or other shape cross section. In some embodiments, the head 804 has a generally square shape adapted to fit within a corresponding head bore 600 of header 402 that has a similar square shape such that when the head 804 is inserted into the head bore 600 the pin 506A,B is prevented from rotating. In other embodiments, the head 804 and head bore 600 have a rectangular, triangular, hexagonal, T, L, or other polygonal or other non-circular shape that provides functionality to prevent the pin from rotating within the bore 502 when inserted. It should be noted that, in FIGS. 5-8, pin 506A is shown in a partially inserted (unlocked) position, and pin 506B is shown in a fully inserted (locked) position.

In embodiments, the connector block includes a first seal 812 in connector block 404 and a second seal 814 positioned in sealing member 802. In one embodiment, first seal 812 is a rigid or semi-rigid plastic cap, or O-ring, or ring shaped gasket, which may comprise rubber, silicone, PEEK or other polymers or materials that allow the device to function as described herein. The first seal 812 is positioned within an undercut section 818 of the connector block 404. The first seal 812 has an inner diameter D_{I} and an outer diameter Dₒ. The outer diameter Dₒ is configured to match, or have an interference fit to, the diameter of the undercut section 818 such that the first seal 812 tightly fits therein and does not substantially shift or rotate during use. In one embodiment, the inner diameter D_{I} has a diameter that is smaller than the outer diameter of the first body portion 806 of pin 506A,B when first seal 812 is in a relaxed (unexpanded) state (i.e., when the pin 506A,B is not inserted therein). In another embodiment, the inner diameter D_{I} has a diameter that is substantially the same as the outer diameter of the first body portion 806 of pin 506A,B when first seal 812 is in an expanded state. Accordingly, when the first body portion 806 is within first seal 812, the first seal 812 seals against first body portion 806 in a liquid tight and/or hermetic sealing manner.

At the distal end of pin 506A,B, the second body portion 808 is fit within second seal 814. Second seal 814 may similar in size, shape and material to first seal 812 or in other embodiments it may be different. For example, in embodiments, second seal 814 may comprise one or more of a rigid or semi-rigid plastic cap, or O-ring, or ring shaped gasket, which may comprise rubber, silicone, PEEK or other polymers or materials that allow the device to function as described herein. In one embodiment, second seal 814 has an inner diameter D₁₂ and an outer diameter D₀₂. The inner diameter D₁₂ in has a diameter that is smaller than the outer diameter of the second body portion 808 of pin 506A,B when the second seal 814 is in a relaxed (unexpanded) state (i.e., when the pin 506A,B is not inserted therein). The outer diameter D₀₂ of second seal 814 is sized to seal within a secondary seal 816 of septum 508. In one embodiment, the second seal 814 is coupled to the second body portion 808 of pin 506A,B such that it is movable from an inactive position Pᵢ outside of septum 508 to an active position Pₐ wherein the second seal is sealingly engaged with secondary seal 816 of septum 508.

In embodiments, the connector block 404 is formed of a non-metallic material, such as PEEK or another biocompatible plastic. In embodiments, the pins 506A,B are formed of PEEK or another biocompatible plastic. However, in other embodiments, the connector block 404 and/or the pins 506A,B may be formed of a metal, metal alloy, polymer, or any other material that allows the device to function as described herein.

Operation of the pins 506A,B will be described with further reference to FIGS. 5-9. In one embodiment, in operation, the pin(s) are placed in the unlocked position as represented by pin 506A. The pin may be placed in the unlocked position by pulling on the head 804 or pushing on the end face 810 of the pin through sealing member 802 until the pin is in the unlocked position. To lock a terminal of the stimulation lead 110 into the connector block 404, a user inserts the terminal into the bore 502 of the connector block 404 until it is fully inserted and the inline contacts are fully seated to the connectors 504. After the terminal is fully seated within the bore 502, the user applies a force to the head 804 of the pin in a direction toward the connector block 404. The applied force should be sufficient to push the pin into the locked position as shown by pin 506B. In the locked position, the pin 506B has first body portion 806 sealingly engaged with first seal 812 and second body portion 808 is sealingly engaged with second seal 814 and second seal 814 is sealingly engaged with secondary seal 816. Such seals may provide air tight, liquid tight or hermetic seals.

In one embodiment, when the pin is in the locked position, as shown as pin 506B, the first body portion 806 of the pin 506B presses against the terminal in an interference fit manner at the location 820. Location 820 is a location at which the pin 506B and the bore 502 (with the terminal inserted therein) intersect. The amount of overlap of the first body portion 806 into the bore 502 may be controlled to provide a desired level of force or friction to prevent the pin from becoming inadvertently removed or dislodged but at the same time ensuring that no damage to the terminal of the stimulation lead 110. In other embodiments, in addition to, or as an alternative to, the interference fit at location 820 the sealing engagement of the second seal 814 with secondary seal 816 and/or the sealing engagement of first seal 812 with first body portion 806 is sufficiently tight as an interference fit to hold the pin in the locked position (pin 506B). In some embodiments, the head 804 has a generally square shape adapted to fit within a corresponding head bore 600 of header 402 that has a similar square shape such that when the head 804 is inserted into the head bore 600 the pin 506A,B is prevented from rotating. In other embodiments, the head 804 and head bore 600 have a rectangular, triangular, hexagonal, T, L, or other polygonal or other non-circular shape that provides functionality to prevent the pin from rotating within the bore 502 when inserted.

To remove a terminal of stimulation lead 110 from the connector block 404 when the terminal is locked into the connector block with pins 506A,B, a user first places the pin 506B into the unlocked pin position (e.g., shown in 506A), using the process described above. After the pin is placed into the unlocked position, the terminal may be pulled out from connector block 404.

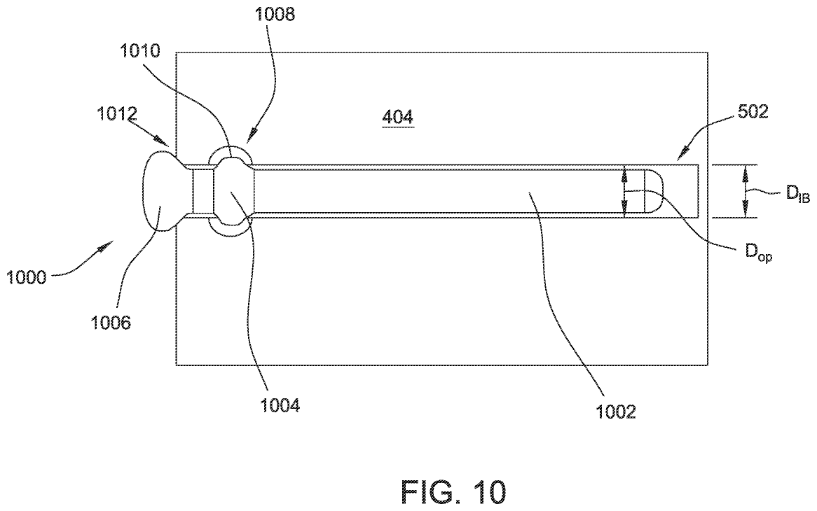

In some embodiments, it may be desirable to remove the terminals of the stimulation leads 110 from the connector block for a period of time. During this time when a lead is not inserted into the bore 502, there is a possibility for tissue or body fluid ingress into the connector block 404 causing undesirable clogging of the bore 502. In order to prevent this undesirable ingress of body fluid or tissue, a port plug 1000 (FIG. 10) may be inserted into the bore 502. In one embodiment, the port plug 1000 includes an elongate body 1002, a shoulder 1004 and a plug head 1006. The port plug 1000 is generally cylindrical in shape. The elongate body 1002 has an outer diameter Dₒₚ that is sized to be the same as, or slightly smaller than the inner diameter D_{IB} of the bore 502. In one embodiment, the bore 502 includes a shoulder engagement section 1008 that has an inner surface sized and shaped to accommodate shoulder 1004 of the port plug 1000. In one embodiment, the shoulder engagement section has an inner face 1010 that has a diameter larger than the inner diameter D_{IB} of the bore 502. The shoulder engagement section 1008 is configured (i.e., appropriately sized and shaped) to provide a snap-fit with the shoulder 1004. Accordingly, when a user inserts the port plug into the bore 502, a snapping sensation provides tactile feedback to the user once the shoulder 1004 is fully seated within the shoulder engagement section 1008.

In some embodiments, the bore 502 does not include a shoulder engagement section 1008, and the bore 502 has a substantially constant inner diameter D_{IB} along its entire length. In this embodiment, once the shoulder 1004 has been pressed through the bore opening 1012, a snapping or tactile feedback is felt by the user, which alerts the user that the shoulder 1004 has been fully seated within the bore 502.

In embodiments, the port plug 1000 may be formed of the same or different material from the connector block 404. For example, in a preferred embodiment, one or both of the connector block 404 and the port plug 1000 comprise PEEK. In one embodiment, the port plug 1000 comprises a radiopaque material sufficient to facilitate determination of its presence and location using X-ray or flouroscopy procedures. In one exemplary embodiment, the port plug 1000 comprises PEEK doped with 20 percent barium sulfate.

In one embodiment, the shoulder 1004 provides a sealing engagement to the bore 502 or the shoulder engagement section 1008 of the bore 502. The sealing engagement of the shoulder 1004 provides a sealing engagement to the bore 502 or the shoulder engagement section 1008 of the bore 502 provides a liquid tight or hermetic seal to prevent undesirable tissue or body fluid ingress into the bore 502. In another embodiment, the plug head 1006 is configured to provide a second sealing engagement to the bore opening 1012. The sealing engagement of the plug head 1006 to the bore opening 1012 may be a liquid tight or hermetic seal to provide an initial barrier to prevent body fluid or tissue ingress into the bore 502.

The following embodiments are provided to illustrate aspects of the disclosure, although the embodiments are not intended to be limiting and other aspects and/or embodiments may also be provided.

Embodiment 1. An implantable pulse generator includes a housing component containing electrical circuitry for generating electrical pulses and a header component connected to the housing component. The header component is adapted to connect to one or more stimulation leads for applying the electrical pulses to the tissue of the patient. The header includes a locking member for an electrical terminal of the implantable pulse generator. The locking member includes a head, an elongate body portion adjacent the head and has a proximal section with a first diameter and a distal section having a second diameter. A transition section is between the proximal section and the distal section. The transition section smoothly transitions from the first diameter to the second diameter. The elongate body portion is configured to provide an interference fit to the electrical terminal in a locked position and hold the electrical terminal in place.

Embodiment 2. The implantable pulse generator according to embodiment 1, the head comprising a non-circular cross sectional shape.

Embodiment 3. The implantable pulse generator according to any prior embodiment, wherein the head is configured to prevent the elongate body from rotating when the locking member is in the locked position.

Embodiment 4. The implantable pulse generator according to any prior embodiment, wherein the locking member comprises PEEK.

Embodiment 5. The implantable pulse generator according to any prior embodiment, wherein the proximal portion is configured to provide a sealing engagement with a first seal of the implantable pulse generator.

Embodiment 6. The implantable pulse generator according to any prior embodiment, wherein the distal portion comprises a second seal configured to provide a sealing engagement with a secondary seal of the implantable pulse generator.

Embodiment 7. A connection system for an implantable medical device (IMD), comprising: a connector block and a plurality of electrical contacts disposed sequentially with a through bore of the IMD, the through bore sized to accept a terminal of a stimulation lead; a septum; a first seal; a locking pin, the locking pin comprising: a head; an elongate body portion adjacent the head and having a proximal section with a first diameter and a distal section having a second diameter; a transition section between the proximal section and the distal section, the transition section smoothly transitioning from the first diameter to the second diameter; wherein the elongate body portion is configured to provide an interference fit to the first seal when in a locked position and hold the terminal in place.

Embodiment 8. The connection system according to embodiment 7, wherein the head comprises a non-circular cross sectional shape.

Embodiment 9. The connection system according to any prior embodiment, wherein the connector block comprises a head bore having a shape corresponding to the non-circular cross sectional shape of the head.

Embodiment 10. The connection system according to any prior embodiment, wherein the proximal section of the elongate body is configured for sealing engagement to the first seal.

Embodiment 11. The connection system according to any prior embodiment, wherein the septum comprises a secondary seal, and the distal section of the elongate body is configured for sealing engagement to the secondary seal.

Embodiment 12. The connection system according to any prior embodiment, wherein the locking pin is configured to provide an interference fit that provides a tactile feedback upon being placed in the locked position.

Embodiment 13. The connection system according to any prior embodiment, wherein the connector block and the locking pin comprise PEEK.

Embodiment 14. The connection system according to any prior embodiment, wherein the locking pin is configured to be placed in an unlocked position by applying a longitudinal force to an end face of the distal section.

Embodiment 15. The connection system according to any prior embodiment, wherein an inner diameter of the first seal is smaller than an outer diameter of the proximal section of the elongate body.

Embodiment 16. A port plug for a connector block of an implantable medical device, comprising: a plug head having a first diameter; an elongate body portion having a second diameter; a shoulder disposed between the plug head and the elongate body portion, the shoulder having a third diameter larger than the second diameter.

Embodiment 17. The port plug according to embodiment 16, wherein the port plug comprises PEEK.

Embodiment 18. The port plug according to any prior embodiment, wherein the port plug is doped with a radiopaque material.

Embodiment 19. The port plug according to any prior embodiment, wherein the first diameter is larger than the second and the third diameters.

Embodiment 20. The port plug according to any prior embodiment, wherein the shoulder is configured to provide a fluid tight sealing engagement to a bore of the implantable medical device.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An implantable pulse generator for generation of electrical pulses to tissue of a patient, comprising:
a housing component containing electrical circuitry for generating electrical pulses;
a header component connected to the housing component, wherein the header component is adapted to connect to one or more stimulation leads for applying the electrical pulses to the tissue of the patient;
wherein the header component comprises:
a locking member for an electrical terminal of the implantable pulse generator, the locking member comprising:
a head;
an elongate body portion adjacent the head and having a proximal section with a first diameter and a distal section having a second diameter;
a transition section between the proximal section and the distal section, the transition section smoothly transitioning from the first diameter to the second diameter; and
wherein the elongate body portion is configured to provide an interference fit to the electrical terminal in a locked position and hold the electrical terminal in place.

2. The implantable pulse generator according to claim 1, the head comprising a non-circular cross sectional shape.

3. The implantable pulse generator according to claim 2, wherein the head is configured to prevent the elongate body portion from rotating when the locking member is in the locked position.

4. The implantable pulse generator according to claim 1, wherein the locking member comprises PEEK.

5. The implantable pulse generator according to claim 1, wherein the proximal section is configured to provide a sealing engagement with a first seal of the implantable pulse generator.

6. The implantable pulse generator according to claim 5, wherein the distal section comprises a second seal configured to provide a sealing engagement with a secondary seal of the implantable pulse generator.

7. A connection system for an implantable medical device (IMD), comprising:
a connector block comprising a plurality of electrical contacts disposed sequentially within a through bore of the IMD, the through bore sized to accept a terminal of a stimulation lead;
a septum;
a first seal;
a locking pin, the locking pin comprising:
a head;
an elongate body portion adjacent the head and having a proximal section with a first diameter and a distal section having a second diameter;
a transition section between the proximal section and the distal section, the transition section smoothly transitioning from the first diameter to the second diameter; and
wherein the elongate body portion is configured to provide an interference fit to the first seal when in a locked position and hold the terminal in place.

8. The connection system according to claim 7, wherein the head comprises a non-circular cross sectional shape.

9. The connection system according to claim 8, wherein the connector block comprises a head bore having a shape corresponding to the non-circular cross sectional shape of the head.

10. The connection system according to claim 7, wherein the proximal section of the elongate body portion is configured for sealing engagement to the first seal.

11. The connection system according to claim 10, wherein the septum comprises a secondary seal, and the distal section of the elongate body portion is configured for sealing engagement to the secondary seal.

12. The connection system according to claim 7, wherein the locking pin is configured to provide an interference fit that provides a tactile feedback upon being placed in the locked position.

13. The connection system according to claim 7, wherein the connector block and the locking pin comprise PEEK.

14. The connection system according to claim 7, wherein the locking pin is configured to be placed in an unlocked position by applying a longitudinal force to an end face of the distal section.

15. The connection system according to claim 7, wherein an inner diameter of the first seal is smaller than an outer diameter of the proximal section of the elongate body portion.

16. A port plug for a connector block of an implantable medical device, comprising:
a plug head having a first diameter;
an elongate body portion having a second diameter; and
a shoulder disposed between the plug head and the elongate body portion, the shoulder having a third diameter larger than the second diameter.

17. The port plug according to claim 16, wherein the port plug comprises PEEK.

18. The port plug according to claim 17, wherein the port plug is doped with a radiopaque material.

19. The port plug according to claim 16, wherein the first diameter is larger than the second and the third diameters.

20. The port plug according to claim 16, wherein the shoulder is configured to provide a fluid tight sealing engagement to a bore of the implantable medical device.
